(19) United States
(12) Patent Application Publication
(10) Pub. No.: US 2022/0061696 A1
(43) Pub. Date: Mar. 3, 2022

(54) OPTICAL IMAGE PHYSIOLOGICAL MONITORING SYSTEM WITH RADAR DETECTION ASSISTANCE

(71) Applicant: Yun yun AI Baby camera Co., Ltd., Taipei City (TW)

(72) Inventors: Chih-Hsin TSENG, Taipei City (TW); Shih-Yun SHEN, Taipei City (TW); Hsin-Yi LIN, Taipei City (TW); Kang-Ning SHAN, Taipei City (TW); Hsueh-Fa HSU, Taipei City (TW); Por-Sau LIN, Taipei City (TW); Chien-Yu CHEN, Taipei City (TW); Tzu Ling LIANG, Taipei City (TW); Huan-Yun WU, Taipei City (TW); Yu-Chiao WANG, Taipei City (TW); Chien-Hui HSU, Taipei City (TW)

(21) Appl. No.: 17/138,527

(22) Filed: Dec. 30, 2020

(30) Foreign Application Priority Data
Sep. 3, 2020 (TW) 109130299

(51) Int. Cl. A61B 5/08; A61B 5/00

(52) U.S. Cl. CPC A61B 5/0823; A61B 5/0013; A61B 5/7264; A61B 5/746; A61B 5/0017

(57) ABSTRACT

An optical image physiological monitoring system with radar detection assistance is disclosed. The system receives a visible-light image of a body and then identifies a chest feature of the visible-light image and determines a position of the chest feature. A processing unit controls a radar detector to move to aim a chest of the body. After then, a plurality of visible-light images and distance values from the radar detector are received continuously to determine whether the positions of the chest features are in a position change rage. If yes, a breathing frequency of the body is determined by the distance values received simultaneously.

### CROSS-REFERENCE TO RELATED APPLICATIONS

This application is based upon and claims priority under 35 U.S.C. 119 from Taiwan Patent Application No. 109130299 filed on Sep. 3, 2020, which is hereby specifically incorporated herein by this reference thereto.

### BACKGROUND OF THE INVENTION

### 1. Field of the Invention

The present invention is related to an optical image physiological monitoring system, and more particularly to an optical image physiological monitoring system with radar detection assistance.

### 2. Description of the Prior Arts

At present, many image monitoring devices or systems have come out to provide remote users real images at local. With the advancement of image processing technology, the image monitoring devices or systems can identify any human body shown in the images and further analyzes the human body's motion. Therefore, the remote user does not necessarily monitor the images from the image monitoring devices or systems to watch people's activities at local.

China Patent Publication No. 110192862 proposes a contactless breathing detection device. The contactless breathing detection device uses a radar detector to detects a human's breath. However, a radius of a detection area of the radar detector is about 5 meters so that the radar detector roughly detects whether any moving subject, such as a human body, is shown in the detection area. Therefore, the breathing detection device is used to determine whether anyone enter or leave a room when the breathing detection device is placed in the room. The breathing detection device also uses the radar detector to detect the human's breath, but many signal-processing technologies are required to extract real radar signals for the human's breath from lots of the reflected radar signals.

To overcome the shortcomings, the present invention provides an optical image physiological monitoring system with radar detection assistance to mitigate or to obviate the aforementioned problems.

### SUMMARY OF THE INVENTION

An objective of the present invention is to provide an optical image physiological monitoring system with radar detection assistance.

To achieve the objective as mentioned above, the optical image physiological monitoring system with radar detection assistance has:

a casing;

a visible-light image sensor mounted on the casing and outputting a plurality of visible-light images of a body;

a radar detector movably mounted on the casing and outputting a plurality of distance values; and

a processing unit mounted in the casing and electrically connected to the visible-light image sensor and the radar detector to receive the visible-light images and the distance values, wherein the processing unit identifies a chest feature of the body from each visible-light image and a position of the chest feature through a deep-learning module; the processing unit controls the radar detector to move relatively to the casing according to the position of the chest feature of the body; and the processing unit has a physiological status determining procedure having:
a learning mode generating a normal breathing frequency of the body; and
a physiological status monitoring mode continuously receiving the visible-light images, continuously receiving the distance values, and continuously determining the positions of the chest features of the visible-light images by a deep-learning module, wherein when the positions of the chest feature are in a position change range, a current breathing frequency is determined according to the distance values obtained simultaneously; the current breathing frequency is compared with the normal breathing frequency to determine whether the current breathing frequency is abnormal; and if the current breathing frequency is abnormal, a physiological monitoring alarm is generated.

With the foregoing description, the visible-light-image physiological monitoring system of the present invention uses the deep-learning module to identify the position of the body's chest feature, so that the processor can control the radar detector to aim the chest of the body. When people breathe, his or her chest alternately expands and shrinks. Since the radar detector transmits the radar signals to the chest of the body and receives the reflected radar signals, the radar detector can calculate different distance values corresponding expanding chest and shrinking chest. Therefore, the processor determines the body's breathing frequency by analyzing a variation of the distance values. Since the processor receives the visible-light images and the distance values simultaneously, the processor determines the body's motion by the deep-learning module and further filters the distance values received under the body with a larger motion to determine the current breathing frequency accurately.

Other objectives, advantages and novel features of the invention will become more apparent from the following detailed description when taken in conjunction with the accompanying drawings.

### BRIEF DESCRIPTION OF THE DRAWINGS

FIG. 1 is a schematic view of a visible-light-image physiological monitoring system mounted on a bedside in accordance with the present invention;

FIG. 2 is a perspective view of the visible-light-image physiological monitoring system in accordance with the present invention;

FIG. 3 is a visible-light image in accordance with the present invention;

FIG. 4A is a functional block diagram of a first embodiment of the visible-light-image physiological monitoring system in accordance with the present invention;

FIG. 4B is a functional block diagram of a second embodiment of the visible-light-image physiological monitoring system in accordance with the present invention;

FIG. 5 is a flow chart of a learning mode in accordance with the present invention;

FIG. 6 is a flow chart of a physiological status monitoring mode in accordance with the present invention; and

FIG. 7 is a distance value vs. time graph in accordance with the present invention.

### DETAILED DESCRIPTION OF THE PREFERRED EMBODIMENTS

With multiple embodiments and drawings thereof, the features of the present invention are described in detail as follows.

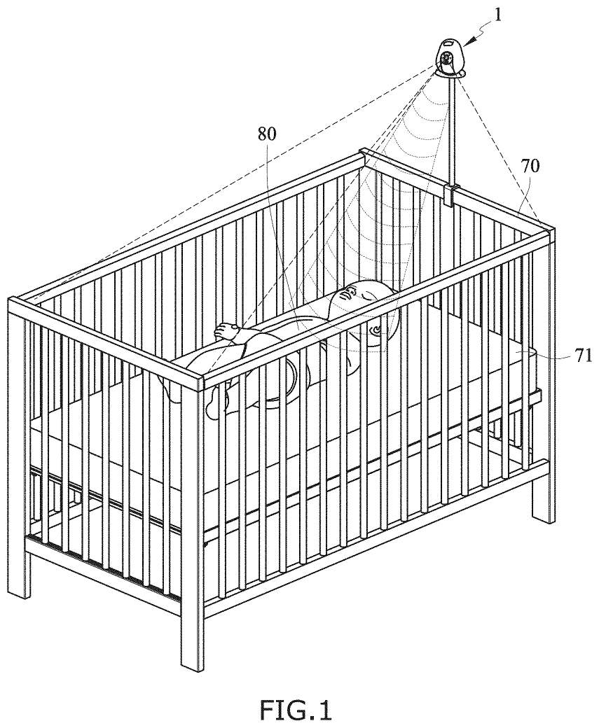

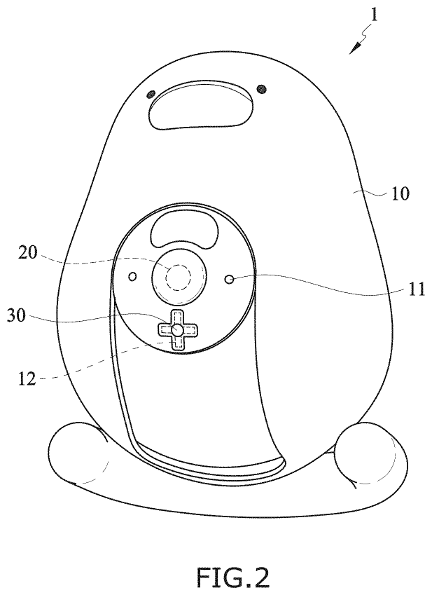

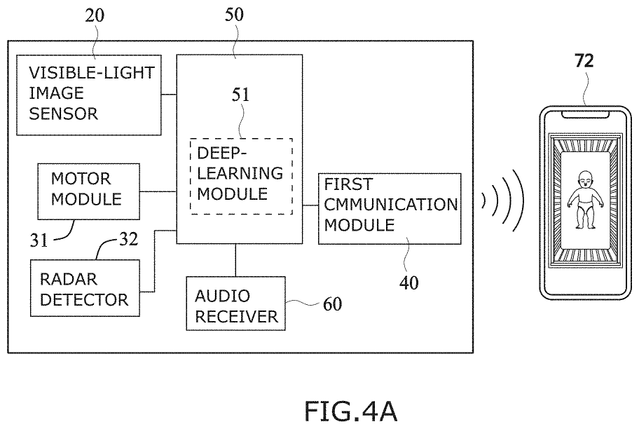

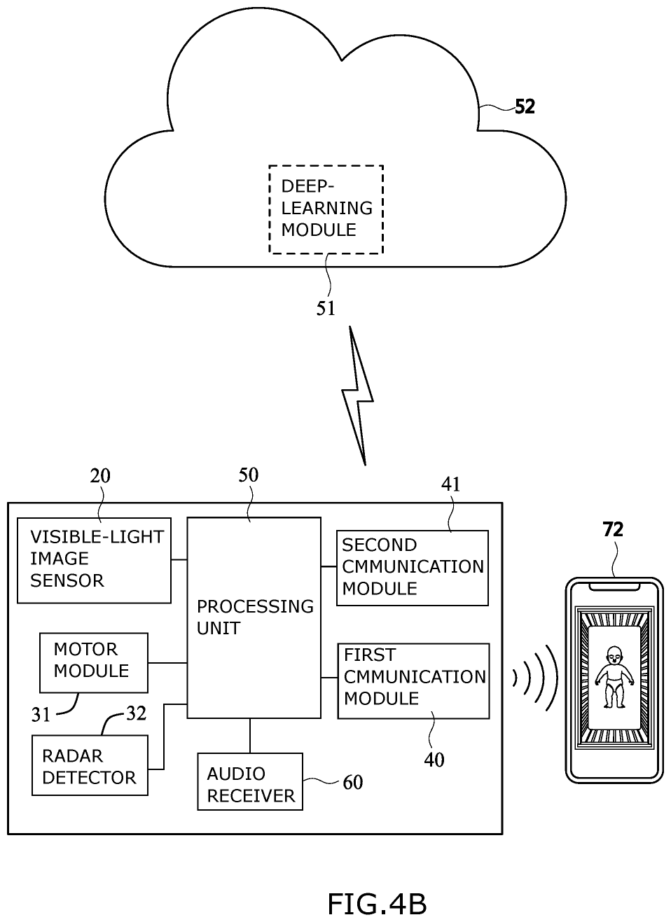

With reference to FIG. 1, a visible-light-image physiological monitoring system 1 of the present invention is a fixed system so that the system may be mounted on a bedside 70 or around a bed to monitor different physiological states of a body 80 on the bed. With further reference to FIGS. 2 and 4A, the system 1 has a casing 10, a visible-light image sensor 20, a radar detector 30 and a processing unit. In one embodiment, the system 1 may further have a first communication module 40 and an audio receiver 60.

In the preferred embodiment, as shown in FIG. 2, at least one through hole 11 is formed through the casing 10 and a dual-shaft moving device 12 is mounted in the casing 10. The radar detector 30 is mounted on the dual-shaft moving device 12 and the audio receiver 60 receives an environmental audio from the through hole 11 and outputs an audio signal.

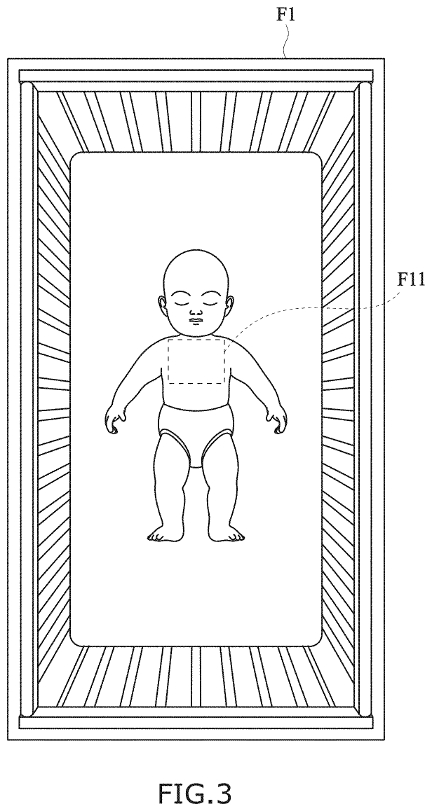

The visible-light image sensor 20 is mounted on the casing 10 and outputs a visible-light image F1, as shown in FIG. 3. The visible-light image sensor 20 aims to the bed and a shooting range of the visible-light image sensor 20 covers a bed surface 71 of the bed. When a body in the bed, the visible-light image sensor 20 outputs the visible-light image F1 of the body.

The radar detector 30 is movably mounted on the casing 10 and outputs a distance value. As shown in FIG. 1, the radar detector 30 aims to the bed. A sensing range of the radar detector 30 is within the bed surface 71. In one embodiment, the radar detector 30 is a mmWave radar detector to output mmWave signals continuously, to receive the reflected mmWave signals and to calculate the distance values. With reference to FIGS. 2 and 4A, the radar detector 30 is mounted on the dual-shaft moving device 12. A motor module 31 is mounted in the casing 10 to connect to the dual-shaft moving device 12. The processing unit 50 drives the motor module 31 to move the dual-shaft device 12 relatively to the casing 10. In one embodiment, the processing unit 50 drives the motor module 31 to move the radar detector 30 to aim the chest 80 of the monitored body. As shown in FIG. 1, the radar detector 30 outputs different distance values corresponding to expanding chest and shrinking chest during breathing.

The first communication module 40 is mounted in the casing 10. In one embodiment, the first communication module 40 matches a wireless communication device 72, such as WIFI or Bluetooth and so on.

The processing unit 50 is mounted in the casing 10 and electrically connected to the visible-light image sensor 20, the radar detector 30 and the audio receiver 60 to receive the visible-light image F1, the distance value and the audio signal. In one embodiment, the processing unit 50 may be an AI processor having a built-in deep-learning module 51. The deep-learning module 51 identifies a chest feature from the visible-light image and further determines a position of the chest feature. The processing unit 50 is electrically connected to the first communication module 40 and transmits a physiological monitoring alarm to the wireless communication device 72 through the first communication module 40. In one embodiment, as shown in FIG. 4B, the visible-light-image physiological monitoring system further has a cloud server 52. The processing unit 50 may link the cloud server 52 through a second communication module 41. The processing unit 50 may upload the received visible-light images to the cloud server 52. The cloud server 52 has a deep-learning module 51 to identify the chest feature of the body from the received visible-light images and the positions of the chest feature. The cloud server 52 sends the processing unit 50 the identified chest feature and the determined positions thereof. The processing unit 50 further has a physiological status determining procedure having a learning mode and a physiological status monitoring mode. The processing unit 50 further determines a decibel value of the received audio signal from the audio receiver 60.

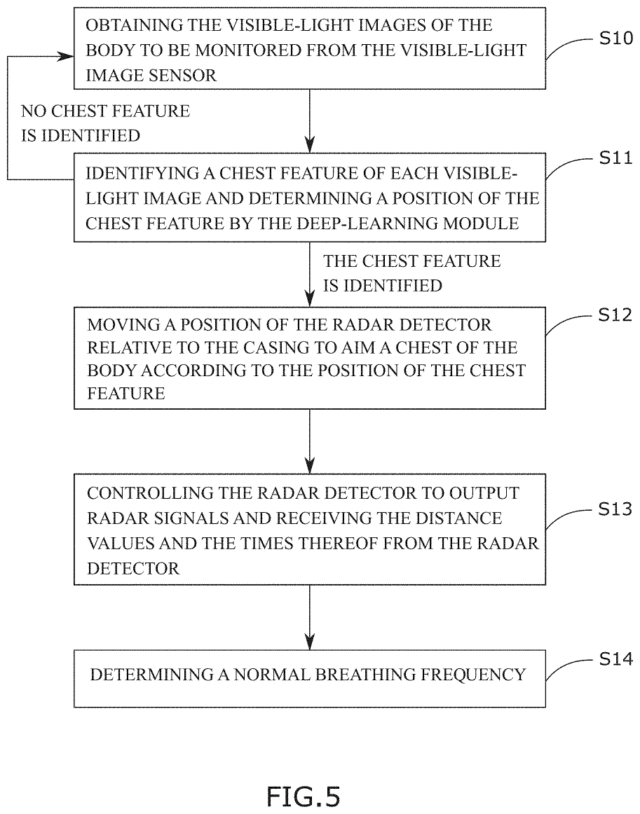

The learning mode generates a normal breathing frequency. With reference to FIGS. 4A and 5, the learning mode has following steps of S10 to S14.

In the step 510, with reference to FIG. 3, the visible-light image F1 of the body to be monitored is obtained from the visible-light image sensor 20. In the step S11, a chest feature F11 of the body to be monitored is identified from the visible-light image F1 and the position of the chest feature F11 is determined by the deep-learning module 51. If no chest feature F11 is identified, go to the step S10. If the chest feature F11 is identified, go to the next step S12. In one embodiment, the deep-learning module 51 identifies a head feature, two hand features, two leg features from the visible-light image F1, determines a pose of the body, and then calculates the position of the chest feature according to relationships among the head feature, the hand features and the leg features and/or pose of the body.

In the step 12, the processing unit 50 adjusts the position of the radar detector 30 by driving the motor module 31 according the position of the chest feature F11 and the radar detector 30 aims the chest 80 of the body to be monitored. In one embodiment, the motor module 31 uses a close-loop-control x-axis server motor system and a close-loop-control y-axis server motor system. The position of the motor module 31 obtains a feedback signal with a rotating angle of the x-axis server motor and a feedback signal with a rotating angle of the y-axis server motor and then determines a coordinate of the radar detector 30 according to the two feedback signals. The coordinates of the radar detector 30 are further corrected by coordinates of a view field of the visible-light image sensor 20, so that a relationship between a detecting range of the radar detector 30 and a shooting range of the visible-light image sensor 20 is obtained by the processing unit 50. Therefore, the processing unit 50 can control the radar detector 30 to accurately aim the chest of the body to be monitored according to the position of the chest feature F11 determined in the step S11.

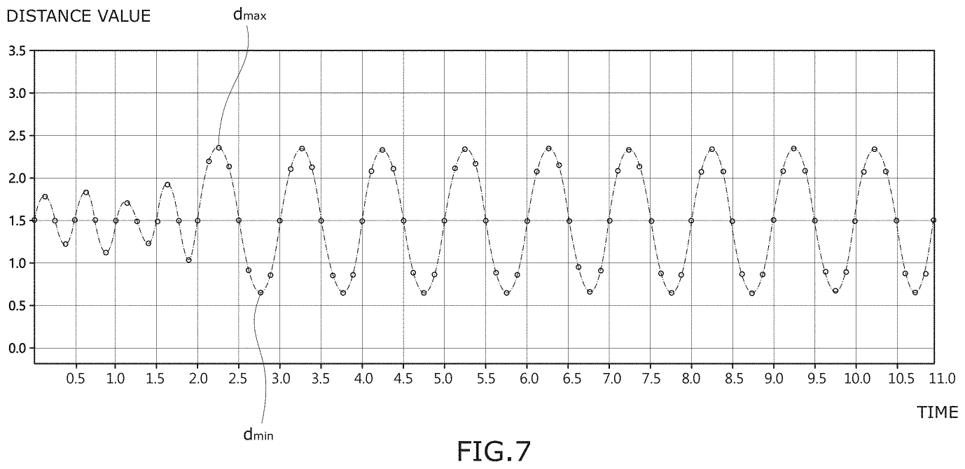

In the step S13, the processing unit 50 controls the radar detector 30 to output the radar signal. With reference to FIGS. 1 and 4A, the radar signal is reflected by the chest 80 and the radar detector 30 calculates the distance value according to a difference between the radar signal and the corresponding reflected radar signal after receiving the reflected radar signal. Therefore, the processing unit 50 receives and stores a plurality of distance values and times thereof. As shown in FIG. 7, using a baby as an example, the normal breathing frequency is about 40˜60 times per minute. If the baby exhales and inhales once in one second, the radar detector 30 has to output at least one distance value in a half of one second. In one embodiment, the radar detector 30 calculates the distance vale based on ToF (time of flight). The time of flight is a time difference between a time of the outputting radar signal and a time of receiving the corresponding received radar signal. The distance value between the reflected surface and the radar detector 30 is calculated based on the time difference. Take a 22 GHz mmWave radar detector as an example, the distance change of mm level within 2 meters can be accurately measured, so the radar detector can be used to detect expanding chest and shrinking chest.

In the step S14, the visible-light image from the visible-light image sensor 20 and the distance value from the radar detector 30 are obtained simultaneously. The deep-learning module 51 determines the positions of the chest feature F11 in a preset period, as shown in FIG. 3. When the positions of the chest feature F11 are in a position change range, the body is static in the preset period. Therefore, the distance values obtained in the preset period, as shown in FIG. 7, are able to be analyzed whether the distance values and the times thereof are changed stably. For example, if the times of the largest distance values dₘₐₓ or the smallest distance values dₘᵢₙ are repeated periodically, a period term is converted to a normal breathing frequency (60 times/min) of the body to be monitored.

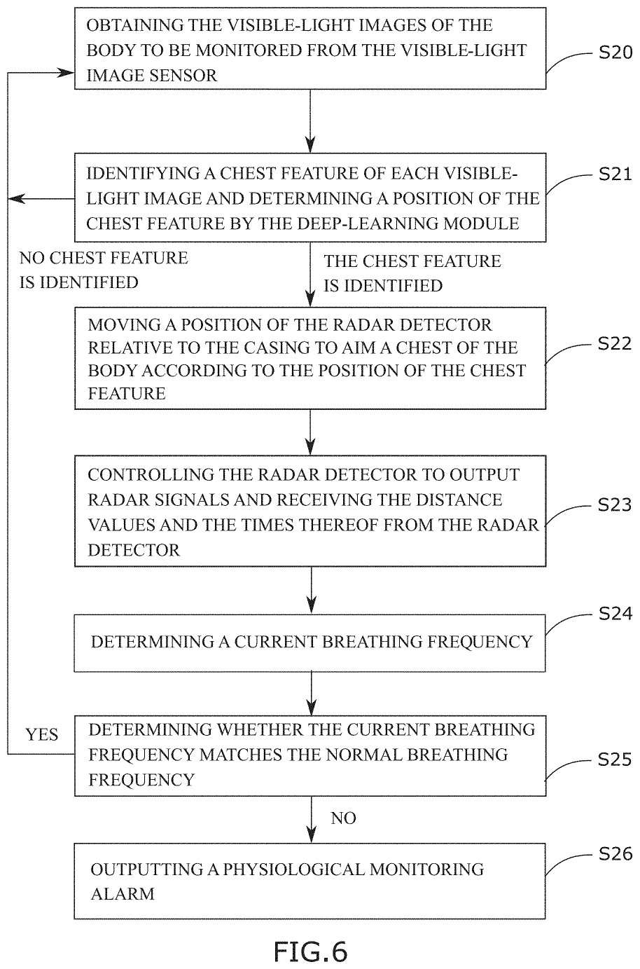

The physiological status monitoring mode is executed after the learning mode is finished. With reference to FIG. 6, in the physiological status monitoring mode, a plurality of visible-light images and a plurality of distance values are continuously received to further determine whether the current breathing frequency is abnormal according to the normal breathing frequency. If the abnormal breathing frequency is abnormal, a physiological monitoring alarm is generated. The physiological status monitoring mode has following steps S20 to S25.

In the step S20, as shown in FIGS. 4A and 4B, a plurality of visible-light images from the visible-light image sensor 20 and a plurality of distance values from the radar detector 30 are continuously received.

In the step S21, as shown in FIG. 3, the chest feature F11 of the visible-light image F1 and a position thereof are determined by the deep-learning module 51. In one embodiment, the deep-learning module 51 determines the chest feature F11 of the visible-light image F1 and the position thereof. Furthermore, the deep-learning module 51 determines the positions of the chest feature F11 in a preset period and whether the positions of the chest feature F11 are in a position change range. If so, the body is static in the preset period and go to the step S22.

In the step S22, according to the position of the chest feature, the position of the radar detector 30 is adjusted to aim the chest of the body to be monitored.

In the step S23, as shown in FIGS. 4A and 4B, the processing unit 50 controls the radar detector 30 to output a radar signal and receives the distance values and the times thereof from the radar detector 30.

In the step S24, as shown in FIG. 7, the distance values obtained in the preset period is analyzed whether the distance values and the times thereof are changed stably. For example, if the times of the largest distance values dₘₐₓ or the smallest distance values dₘᵢₙ are repeated periodically, a period term is converted to a current breathing frequency of the body to be monitored.

In the step S25, the processing unit 50 determines whether the current breathing frequency matches the normal breathing frequency. If yes, the body's breath is normal and goes to the step S20. If not, in step S26, the processing unit 50 outputs the physiological monitoring alarm, including abnormal breath.

In addition, if the abnormal breath is determined in the step S25, the processing unit 50 calculates the decibel value of the received audio signal from the audio receiver 60 and determines whether the decibel value exceeds a preset decibel value. In the baby monitor application, the body to be monitored is the baby's body, and the baby may have a fever and is crying or coughing if the decibel value exceeds the preset decibel value. The processing unit 50 transmits a coughing alarm or crying alarm.

Based on the foregoing description, the visible-light-image physiological monitoring system of the present invention uses the deep-learning module to identify the position of the chest feature of the body, so the processor can control the radar detector to aim the chest of the body. When people breathe, his or her chest alternately expands and shrinks. Since the radar detector transmits the radar signals to the chest of the body and receives the reflected radar signals, the radar detector can calculate different distance values corresponding expanding chest and shrinking chest. Therefore, the processor determines the breathing frequency of the body by analyzing a variation of the distance values. Since the processor receives the visible-light images and the distance values simultaneously, the processor determines body's motion by the deep-learning module and further filters the distance values received under the body with a larger motion to determine the current breathing frequency accurately.

Even though numerous characteristics and advantages of the present invention have been set forth in the foregoing description, together with the details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A optical image physiological monitoring system with radar detection assistance, comprising:
a casing;
a visible-light image sensor mounted on the casing and outputting a plurality of visible-light images of a body;
a radar detector movably mounted on the casing and outputting a plurality of distance values; and
a processing unit mounted in the casing and electrically connected to the visible-light image sensor and the radar detector to receive the visible-light images and the distance values, wherein the processing unit identifies a chest feature of the body from each visible-light image and a position of the chest feature through a deep-learning module; the processing unit controls the radar detector to move relatively to the casing according to the position of the chest feature of the body, and the processing unit has a physiological status determining procedure having:
a learning mode generating a normal breathing frequency of the body; and
a physiological status monitoring mode continuously receiving the visible-light images, continuously receiving the distance values, and continuously determining the positions of the chest features of the visible-light images by a deep-learning module, wherein when the positions of the chest feature are in a position change range, a current breathing frequency is determined according to the distance values obtained simultaneously; the current breathing frequency is compared with the normal breathing frequency to determine whether the current breathing frequency is abnormal; and if the current breathing frequency is abnormal, a physiological monitoring alarm is generated.

2. The optical image physiological monitoring system with radar detection assistance as claimed in claim 1, wherein the learning mode of the physiological status determining procedure has steps of:
(a1) receiving the visible-light image from the visible-light image sensor;
(a2) identifying the chest features of the visible-light images and determining the positions of the chest features by the deep-learning module;
(a3) moving a position of the radar detector relatively to the casing to aim a chest of the body according to the position of the chest feature;
(a4) controlling the radar detector to output radar signals and receiving the distance values and the times thereof from the radar detector; and
(a5) receiving the visible-light images from the visible-light image sensor and the distance values from the radar detector simultaneously; determining the positions of the chest feature in a preset period by the deep-learning module and whether the positions of the chest feature in the preset period, wherein when the positions of the chest feature are in the position change range, the distance values obtained in the preset period are analyzed to determine the normal breathing frequency if the distance values and the times thereof are changed stably.

3. The visible-light-image physiological monitoring system as claimed in claim 2, wherein in the step (a5), when largest distance values or smallest distance values are repeated periodically, a period term is converted to the normal breathing frequency.

4. The visible-light-image physiological monitoring system as claimed in claim 3, wherein the physiological status monitoring mode of the physiological status determining procedure has steps of:
(b1) receiving the visible-light images from the visible-light image sensor;
(b2) identifying the chest features of the visible-light images and determining the positions of the chest features by the deep-learning module;
(b3) moving the position of the radar detector relatively to the casing to aim the chest of the body according to the position of the chest feature of the body;
(b4) controlling the radar detector to output radar signals and receiving the distance values and the times thereof from the radar detector; and
(b5) receiving the visible-light images from the visible-light image sensor and the distance values and the times of the distance values from the radar detector simultaneously; determining the positions of the chest feature in the preset period by the deep-learning module and whether the positions of the chest feature in the preset period, wherein when the positions of the chest feature are in the position change range, the distance values obtained in the preset period are analyzed to determine the current breathing frequency if the distance values and the times thereof are changed stably; and
(b6) determining whether the current breathing frequency matches the normal breathing frequency, wherein if yes, go to the step (b1); and if not, the processing unit outputs the physiological monitoring alarm including abnormal breath.

5. The visible-light-image physiological monitoring system as claimed in claim 4, wherein in the steps (a2) and (b2), the deep-learning module identifies a head feature, two hand features, two leg features from the visible-light image, determines a pose of the body, and then calculates the position of the chest feature according to relationships among the head feature, the hand features and the leg features and the pose of the body.

6. The visible-light-image physiological monitoring system as claimed in claim 4, further comprising a first communication module, wherein in the step (b6), the processing unit transmits the physiological monitoring alarm through the first communication module.

7. The visible-light-image physiological monitoring system as claimed in claim 5, further comprising a first communication module, wherein in the step (b6), the processing unit transmits the physiological monitoring alarm through the first communication module.

8. The visible-light-image physiological monitoring system as claimed in claim 4, wherein
the radar detector is a mmWave radar detector; and
in the steps (a4) and (b4), a time difference between a time of outputting radar signal and a time of receiving the corresponding received radar signal is calculated and the distance value between the chest of the body and the radar detector is calculated based on the time difference.

9. The visible-light-image physiological monitoring system as claimed in claim 5, wherein
the radar detector is a mmWave radar detector; and
in the steps (a4) and (b4), a time difference between a time of outputting radar signal and a time of receiving the corresponding received radar signal is calculated and the distance value between the chest of the body and the radar detector is calculated based on the time difference.

10. The visible-light-image physiological monitoring system as claimed in claim 4, further comprising:
a dual-shaft device mounted on the casing on which the radar detector is mounted; and
a motor module mounted in the casing, electrically connected to the processing unit and connected to the dual-shaft device, wherein the processing unit drives the motor module to move the radar detector through the dual-shaft device.

11. The visible-light-image physiological monitoring system as claimed in claim 5, further comprising:
a dual-shaft device mounted on the casing on which the radar detector is mounted; and
a motor module mounted in the casing, electrically connected to the processing unit and connected to the dual-shaft device, wherein the processing unit drives the motor module to move the radar detector through the dual-shaft device.

12. The visible-light-image physiological monitoring system as claimed in claim 10, wherein
the motor module comprises a close-loop-control x-axis server motor system and a close-loop-control y-axis server motor system, wherein the close-loop-control x-axis server motor system outputs a feedback signal including rotating angle of motor and the close-loop-control y-axis server motor system respectively outputs a feedback signal including rotating angle of motor; and
in the steps (a3) and (b3), a coordinate of the radar detector is determined according the two feedback signals and the coordinate of the radar detector is further corrected by coordinates of a view field of the visible-light image sensor, so a relationship between a detecting range of the radar detector and a shooting range of the visible-light image sensor is obtained by the processing unit, wherein the processing unit controls the radar detector to accurately aim the chest of the body according to the position of the chest feature determined in the steps (a2) and (b2).

13. The visible-light-image physiological monitoring system as claimed in claim 11, wherein
the motor module comprises a close-loop-control x-axis server motor system and a close-loop-control y-axis server motor system, wherein the close-loop-control x-axis server motor system outputs a feedback signal including rotating angle of motor and the close-loop-control y-axis server motor system respectively outputs a feedback signal including rotating angle of motor; and
in the steps (a3) and (b3), a coordinate of the radar detector is determined according the two feedback signals and the coordinate of the radar detector is further corrected by coordinates of a view field of the visible-light image sensor, so a relationship between a detecting range of the radar detector and a shooting range of the visible-light image sensor is obtained by the processing unit, wherein the processing unit controls the radar detector to accurately aim the chest of the body according to the position of the chest feature determined in the steps (a2) and (b2).

14. The visible-light-image physiological monitoring system as claimed in claim 4, wherein the processing unit is electrically connected to an audio receiver to receive and process an audio signal to a decibel value.

15. The visible-light-image physiological monitoring system as claimed in claim 5, wherein the processing unit is electrically connected to an audio receiver to receive and process an audio signal to a decibel value.

16. The visible-light-image physiological monitoring system as claimed in claim 14, wherein in the step (b6), after the abnormal breathing frequency is determined, the decibel value is received to be further determined whether the decibel value exceeds a preset decibel value, wherein if a determining result is positive, a crying alarm or a coughing alarm is transmitted.

17. The visible-light-image physiological monitoring system as claimed in claim 15, wherein in the step (b6), after the abnormal breathing frequency is determined, the decibel value is received to be further determined whether the decibel value exceeds a preset decibel value, wherein if a determining result is positive, a crying alarm or a coughing alarm is transmitted.

18. The visible-light-image physiological monitoring system as claimed in claim 1, wherein the deep-learning module is built in the processing unit.

19. The visible-light-image physiological monitoring system as claimed in claim 1, further comprising:
a second communication module mounted in the casing and electrically connected to the processing unit; and
a cloud server linking to the processing unit through a second communication module and the deep-learning module is built in the cloud server to identify the chest features of the visible-light images and determine the position of each chest feature; wherein the cloud server sends the processing unit the chest features of the visible-light images and the position of each chest feature.

20. The visible-light-image physiological monitoring system as claimed in claim 17, further comprising:
a second communication module mounted in the casing and electrically connected to the processing unit; and
a cloud server linking to the processing unit through a second communication module and the deep-learning module is built in the cloud server to identify the chest features of the visible-light images and determine the position of each chest feature; wherein the cloud server sends the processing unit the chest features of the visible-light images and the position of each chest feature.
